(19) United States
(12) Patent Application Publication
(10) Pub. No.: US 2006/0211660 A1
(43) Pub. Date: Sep. 21, 2006

(54) Combination therapy for topical application in the treatment of dry eye syndrome

(76) Inventors: Charles P. Du Mee, Foster City, CA (US); Gene Barnett, San Francisco, CA (US); Michael Coy, Honolulu, HI (US)

Correspondence Address: REED SMITH LLP, 1301 K STREET, N.W., SUITE 1100 EAST TOWER, WASHINGTON, DC, 20005, US

(21) Appl. No.: 11/365,876

(22) Filed: Mar. 2, 2006

Related U.S. Application Data
(60) Provisional application No. 60/657,404, filed on Mar. 2, 2005.

(51) Int. Cl. A61K 31/66; A61K 31/57; A61K 31/56

(52) U.S. Cl. 514/130; 514/170

(57) ABSTRACT

A topical ophthalmic composition comprising 17-β-estradiol or its derivatives and an androgen in pharmaceutically acceptable vehicle, and method of using same for the alleviation of kerato-conjunctivitis sicca KCS (dry eye syndrome DES).

### FIELD OF THE INVENTION

This invention relates to the topical application of a combination of sex steroids for the treatment of human dry eye syndrome, also known as keratoconjunctivitis sicca (KCS), and more specifically, to the preparation and topical application of androgen analogues and estrogen analogues, such as 17-β-estradiol, and their derivatives in lipid, liposomes, polymers, or aqueous or non-aqueous vehicles. This invention may also be useful in treating other conditions where dry eye syndrome may occur, such as post-operative corneal transplant patients.

### BACKGROUND OF THE INVENTION

Broadly speaking, dry eye syndrome is a disorder of the tear film due to tear deficiency or excessive tear evaporation which causes damage to the interpalpebral ocular surface and is associated with symptoms of ocular discomfort. (M. A. Lemp. Report of the National Eye Institute/Industry Workshop on Clinical Trials in Dry Eyes, The Contact Lens Association of Ophthalmologists Journal, 21(4):221-231 (1995)). Findings show differences between Sjögren's associated keratoconjunctivitis sicca (KCS) and non-Sjögren's KCS. (J. D. Nelson, et al., Cellular Acetate Impressions of the Ocular Surface: Dry Eye States, Arch. Ophthalmol., 101:1869-1982 (1983); S. C. G. Tseng, Staining of Conjunctival Aquamous Metaplasia by Impression Cytology, Ophthalmol., 92:728-733 (1985); S. C. Pflugfelder, et al., Cytological Features of Primary Sjögren's Syndrome, Ophthalmol., 97:985-991 (1990). Neurotransmitters (A. K. Mircheff, et al., Autoimmunity of the Lazcrimal Gland in the Dry Eye, Internat. Ophth. Clinics, 34(1):1-18 (1994); A. K. Mircheff, et al., Understanding the Causes of Lacrimal Insufficiency: Implications for Treatment and Prevention of Dry Eye Syndrome, Res. Prev. Blindness Sci. Writers' Seminar, 51-54. (1993)), viruses (S. C. Pflugfelder, et al., Epstein-Barr Virus and the Lacrimal Gland Pathology of Sjögren's Syndrome, in: Lacrimal Gland, Tear Film and Dry Eye Syndromes, Advances in Exp Med Bid 350, (Sullivan D A., ed., New York, Plenum Press, 1994), pp 641-646), and hormones (D. W. Warren, Hormonal Influences on the Lacrimal Gland in the Dry Eye, Internat. Ophthalmol, Clinics, 47:19-266 (1994); D. A. Sullivan, Ocular Mucosal Immunity, Handbook of Mucosal Immunology (Academic Press, 1994), 47:569-597) are important in regulating tear production and immune activity in the lacrimal glands and the ocular surface. Also, meibomian gland dysfunction can increase tear evaporation with an increase in tear film osmolarity and resultant ocular surface disease. (W. P. Mathers, et al., Meibomian Gland Dysfunction in Chronic Blepharitis, Cornea, 11:763-765 (1991).

Tear film quality depends on fine regulatory mechanisms affected by neuronal and hormonal influences. Indeed, receptors for androgens, estrogens, progesterone and prolactin have been identified in several ocular tissues in the rat, rabbit and in humans. These hormones regulate the immune system, the morphology and secretory functions of lacrimal glands and the functioning of Meibomian glands. The influence of hormone replacement therapy in menopausal women remains unclear, as some authors support the idea that they improve the quality and the volume of tear film, whereas others have argued that they increase the risk of dry eye. Finally, knowledge of the interactions between the hormones that influence the lacrimal glands is essential for the understanding of the regulation of lacrimal gland function. Additional data suggest that optimal bioavailable androgen levels are essential for normal lacrimal gland function and that prolactin and estrogens also play important roles in providing a hormonal milieu that contributes to normal lacrimal gland function. (L. Oprea, A. Tiberghien A., C. Cruezot-Garcher, C. Baudouin, Hormonal Regulatory Influence in Tear Film, J. Fr. Ophthalmol., Oct. 2004; 27(8):933-41 (2004)).

Topical application of androgens or their analogues to patients with KCS, or autoimmune diseases, especially as manifested in Sjögren's syndrome, can directly suppress the immunopathological defects in accessory lacrimal tissue and the main lacrimal gland's palpebral lobe, which is adjacent to the ocular surface. Furthermore, topical androgen treatment can increase both the production and secretion of lipids to reduce meibomian gland dysfunction. (Sullivan, DA, U.S. Pat. No. 6,107,289; Aug. 22, 2000).

The standard treatment of KCS with artificial lubricants, which provides temporary symptomatic relief in most cases does not, however, address the cause of the dry eyes. While there has been described treatment of post menopausal females with dry eye syndrome using oral Premarin therapy, the oral or parenteral administration of estrogen can frequently produce side effects such as vaginal bleeding, breast tenderness and other undesired effects and the therapeutic effects derived from oral therapy are minimal. This is now understood to result from the fact that there are very few estrogen receptors in the conjunctiva relative to other tissues of the body. (Gans, L. A., et al., Estrogen and Progeesterone Recepetors and Human Conjunctiva, Am. J. Ophthalmol. 109(4):474-477 (1990)). Further, such oral or parenteral administration implicates the entire body structure in an indeterminate effort to secure an effect in a localized area (the eye). Conservative medicine would indicate the desirability of limiting the specific effect of the hormone to the recipient site if possible.

One possible method of accomplishing this is through the use of topically applied steroids in drop form. Sator et al. demonstrated that topical estrogen is useful in treating Kerotoconjuntivitis sicca (Sator, et al., Treatment of Menopausal Keratoconjunctivitis Sicca with Topical Oestradiol, Br. J. Obstset. Gynaecol.,105(1):100-2 (1998.)). Addditionally, U.S. Pat. No. 6,096,733, teaches the use of 17-β-estradiol and its derivatives in the treatment of dry eye syndrome. Further, U.S. Pat. No. Re. 34,578 showed that treatment of dry eye syndrome or KCS was shown to be effective using a form of estrogen in solution at concentrations of at least 0.1 mg/ml or 0.1% (w/w).

Further studies since about 1990 have shown that estrogen is a component of human tears and that it may play a role in ophthalmic changes in ocular tissue (Kramer, P. et al., Cyclic Changes in Conjunctival Smears from Menstruating Females, Ophthalmol,. 1990 97:303-307; Metka, M. et al., Ophthalmic complaints as a climacteric symptom, Maturitas, 1991 14:3-8). Other studies, even more recently, have intimated that post-menopausal patients given low systemic doses of estriol (a hydroxylated form of 17-β-estradiol) at a dose of 0.25 mg per day, or that even near homeopathic concentrations of 17-β-estradiol (0.00025%) in drops applied every 6 hours (in women already taking 2 mg estriol valerate daily by mouth) gave varying or marginal improvement in corneal lens transmittance and autofluorescence (Benitez de Castillo, et al., Effects of Estrogen Use on Lens Transmittance in Postmenopausal Women, Ophthalmol., 1997 104:970-973).

Further, U.S. Pat. No. 6,107,289 teaches an approach for management of KCS, especially as manifested in Sjögren's syndrome, involving the topical application to the eye of a preparation containing a therapeutic amount of an androgen or androgen analogue, at a dose rate of less than 1 mg/day. Presently there is no method for treating dry eye syndrome which may be due to an overlap of etiological factors or unknown etiological origin.

The present invention views dry eye syndrome as due to the interaction of numerous factors all or some of which may be so concurrently present in an affected eye to varying degrees, that a combination therapy involving the use of 17-β-estradiol and androgens or androgen analogues (hereinafter collectively referred to as “androgens”) is effective in the management of both Sjögren and non-Sjögren KCS, and in certain cases will have synergistic effect compared to the topical administration of either estrogen or androgen standing alone.

The present invention thus provides a combination therapy for alleviating the symptoms of dry eye syndrome comprising the topical application of an effective amount of 17-β-estradiol analogues and androgens in solution or suspension.

Moreover, one can also significantly decrease any potential systemic absorption of the “hormones,” following topical ophthalmic delivery of the present invention, by combining the use of the drops with a punctal plug. A punctal plug is a small device which fits inside the punctum lacrimale of the eye and prevents tears from draining into the nasopharyngeal cavity through the lacrimal canaliculi. The result of using such a plug is that the tears do not drain away from the corneal surface allowing a greater buildup of lacrimal fluid around the eye. Use of such a plug can either be temporary or permanent and has been used to alleviate eye dryness in patients.

Furthermore, dry eye syndrome also manifests itself in pre-menopausal women who have hormonal abnormalities including insufficient estrogen production. Typically, these patients often present complaints to their ophthalmologists about the inability to wear contact lenses because of their extreme discomfort. Depending on their hormonal profiles, a combination estrogen-androgen topical application may be more effective in the management of dry eye syndrome than either kind of hormone alone.

### SUMMARY OF THE INVENTION

Accordingly, it is a principal object of this invention to provide methods and pharmaceutical compositions comprising estrogen esters and androgens for the treatment of dry eye syndrome or KCS by topical application to the conjunctival surface of the eye. The compositions useful in the invention preferably contain an estrogen analogue, such as 17-β-estradiol, or its esters or salts, such as 17-β-estradiol-3-phosphate, in combination with one or more androgens suspended or dissolved in a suitable vehicle. Suitable vehicles may comprise a lipid (oil based) suspension or an aqueous solution having a pH within the range of 4-8, preferably pH 6-8. It is contemplated that this invention can also utilize a liposomal delivery vehicle as well.

The present invention can advantageously be used to treat symptoms of dry eye syndrome in post-menopausal women, women who have had oophorectomies or total hysterectomies or premature ovarian failure, and pre-menopausal women with hormonal abnormalities including insufficient estrogen production.

Useful androgens for the purposes of this invention include 17-α-methyl-17-β-hydroxy-2-oxa-5α-androstan-3-one, 4,5α-dihydrotestosterone derivatives, testosterone derivatives, 19-nortestosterone derivatives, 17β-hydroxy-5α-androstane derivatives containing ring A unsaturation, their esters, and their cationic or phosphorylated derivatives, designed to increase solubility in hydrophilic media.

It is to be understood that both the foregoing general description and the following detailed description are exemplary and explanatory, and are not intended to limit the invention as claimed. Other objects and features of the invention will become apparent from the following detailed description. All references cited in the instant disclosure are incorporated herein by reference.

### DETAILED DESCRIPTION OF THE PREFERRED EMBODIMENTS OF THE PRESENT INVENTION

The present invention provides a method for the treatment of Dry Eye Syndrome by direct application of compositions containing an estrogen analogue and an androgen in proximity to the conjunctival surface of the eye. Accordingly, in the method of the invention, the therapeutically active agents are applied locally to the site where they are needed, rather than being systemically delivered throughout the body. This provides numerous advantages, including the flexibility to tailor the dose for maximum effect with reduced concern for triggering unwanted side effects in other parts of the body. Consequently, topical administration, according to the invention, may permit the use of higher localized doses with reduced side effects, which can enhance the effectiveness of the treatment as well as patient compliance.

The compositions useful in the invention may contain any therapeutically effective estrogen analogue, including esters and salts thereof. In a preferred embodiment, the formulation comprises a derivative of estrogen known as 17-β-estradiol (or the 3-phosphate disodium salt) or its water-soluble, storage-stable derivatives (beta-estradiol glucuronide, beta-estradiol hemisuccinate, beta-estradiol phosphate, beta-estradiol sulfate and their 3,17 diesters, 17 monoesters and 3 monoesters). The 17-β-estradiol 3-phosphate disodium salt is generally preferred because of the enhanced aqueous solubility and stability of the particular derivative at essentially neutral pH 6-8 (though the pH is not critical and can suitably range between 4-8) and the ease of sterile ophthalmic solution manufacture. The drug substance is also known as 17-β-estradiol 3-phosphate disodium and 1,3,5 (10)-estratriene-3,17 beta-diol 3-phosphate disodium. The formulation is C₁₈H₂₃O₅P₁Na₂, having a molecular weight of 396.3 (anhydrous).

Each gram of 17-β-estradiol (as the 3-phosphate disodium salt) contains approximately 687 milligram of 17-β-estradiol on an anhydrous basis. 17-β-estradiol (as the 3-phosphate disodium salt) is available commercially, such as from Research Plus, Inc., Bayonne, N.J. 07002 (catalog No.1850-5). Particularly preferred is the GMP grade manufactured by Organics LaGrange, Northbrook, Ill. 60062. The compound is a white crystalline powder with an ill-defined melting point and purity better than 97%. The material is to be stored in sealed vials under refrigeration when not in use.

Similarly, the compositions useful in the invention may contain any therapeutically effective androgen, including esters and salts thereof. Selection of the most appropriate therapeutic androgen will depend upon a given hormone's activity, potential side effects and form of administration. For example, topical testosterone may be quite effective in reducing lacrimal inflammation, and its methylated analogue appears to have no toxic side effects on parameters such as intraocular pressure (P. A. Knepper, J. A. Collins, and R. Frederick, Effect of Dexamethasone, Progesterone, and Testosterone on IOP and GAGs in the Rabbit Eye, Invest. Ophthalmol. Vis. Sci. 26:1093-1100 (1985). However, a variety of other modified and/or anabolic androgens (J. D. Wilson and D. W. Foster, eds., Williams Textbook of Endocrinology, WB Saunders Company, Philadelphia (1985), Vida, J. A., “Androgens and Anabolic Agents,” Academic Press, New York (1969)) may be more effective than testosterone. In addition, with regards to administration, if the steroids are to be complexed to a carrier vehicle (e.g., hyaluronate), then a nitrogenated analogue might be indicated or a phosphorylated analogue if an aqueous solution is desired.

In preferred embodiments, the composition comprises an androgen selected from the group consisting of 17-α-methyl-17-β-hydroxy-2-oxa-5α-androstan-3-one, 4,5α-dihydrotestosterone derivatives, testosterone derivatives, 19-nortestosterone derivatives, 17β-hydroxy-5α-androstane derivatives containing ring A unsaturation, their esters, and their cationic or phosphorylated derivatives, designed to increase their solubility in hydrophilic media.

Suitable androgens for use in the invention include testosterone, dihydrotestosterone (also termed allodihydrotestosterone, androstanolone, stanolone, 5 alpha-dihydrostestosterone), fluoxymesterone, stanozolol, nortestosterone propionate, dehydroepiandrosterone (an androgen precursor, also termed androstenolone, dehydroisoandro-sterone, DHEA, transdehydroandrosterone), oxandrolone; methyidihydrotestosterone (also termed methylandrostanolone), oxymetholone, 5 alpha-androstan-17β-ol-3-oxime, 5 alpha-androstan-17 alpha-ol-3-one-acetate, (1) 2,(5 alpha)-androsten-17β-ol, 5 alpha-androstan-2 alpha-methyl-17β-ol-3-one, and methyltestosterone, and their derivatives and esters.

These androgens are representative of the major structural subclasses of androgens, as disclosed in Vida (Vida, J. A., “Androgens and Anabolic Agents,” Academic Press, New York (1969)), herein incorporated by reference. The subclasses include (a) androgenic compounds with unusual structural features (e.g., 17 alpha-methyl-17β-hydroxy-2-oxa-5 alpha-androstan-3-one, also termed oxandrolone); (b) testosterone derivatives (e.g., methyltestos-terone); (c) 4,5 alpha-dihydrotestosterone derivatives (oxymetholone); (d) 17β-hydroxy-5 alpha-androstane derivatives containing a ring A unsaturation, excluding testosterone derivatives (e.g., 2,(5 alpha)-androsten-17β-ol); and (e) 19-nortestosterone derivatives (e.g., 19-nortestosterone propionate).

Also, relative to standards (typically testosterone), these androgens include compounds displaying: (a) augmented androgenic (i.e., virilizing) activity coupled with an even larger increase in anabolic activity (e.g., fluoxymesterone); (b) enhanced anabolic action with unchanged androgenic effects (e.g., oxymetholone, dihydrotestosterone); (c) decreased androgenic ability with unchanged anabolic activity (e.g., 19-nortestosterone propionate); and (d) decreased androgenic capacity paralleled by increased anabolic activity (e.g., oxandrolone, stanozolol).

Preferred androgens of this invention are those which have far more anabolic, than virilizing effect, (e.g., oxandrolone possesses 322% of the anabolic and 24% of the androgenic activity of methyltestosterone (Vida, J. A., “Androgens and Anabolic Agents,” Academic Press, New York (1969)). Particularly preferred androgens are 17-α-methyl-17-β-hydroxy-2-oxa-5α-androstan-3-one, 4,5α-dihydrotestosterone, their esters and phosphorylated derivatives.

Further preferred androgens are nitrogen-substituted androgens such as 5 alpha-androstan-17β-ol 3-oxime, which is created by the substitution of a nitrogen derivative for the 3-ketone function in dihydrotestosterone (very potent androgen) (Vida, J. A., “Androgens and Anabolic Agents,” Academic Press, New York (1969)). This substitution does not inhibit androgen activity (Vida, J. A., “Androgens and Anabolic Agents,” Academic Press, New York (1969)) and may permit binding to hyaluronate for topical administration. Of interest, a variety of other nitrogenated androgens have been shown to express increased anabolic but decreased androgenic activity. These compounds typically contain 3-substitutions, but not nitrogen incorporation in the steroid ring structure, which appears to abolish androgen action (Vidal J. A., “Androgens and Anabolic Agents,” Academic Press, New York (1969)).

In order to increase the aqueous solubility of the androgenic agents, phoshorylated ester derivatives of the androgens are preferred and can be prepared by means commonly available in the art. For example, the most convenient method of synthesis of steroid esters is reaction of the steroid in a 2:1 mixture of pyridine and the anhydride of the desired ester: for example, propionic anhydride would be used to make the propionate ester. A large excess (at least 10 times) of the anhydride compared to the steroid would be required. This would then be purified by diluting with at least 10 parts of water to each part of pyridine, adding 1 part ether, decanting the water after shaking, and then washing with 10 parts water repeatedly in a separatory funnel. This would be followed preferably by recrystallization or chromatography for purification.

The estrogen and androgen compositions may be formulated and applied separately to the eyes in the method of the present invention, or they may be formulated and applied as a single composition. Preferably, they are formulated and applied as a single composition. The preferred embodiment or formulation comprises a solution, suspension or cream of a derivative of estrogen known as 17-β-estradiol (or the 3-phosphate disodium salt) and an androgen preferably selected from the group comprising 17-α-methyl-17-β-hydroxy-2-oxa-5α-androstan-3-one, 4,5α-dihydrotestosterone, and their esters and phosphorylated derivatives.

The amount of active ingredient that is to be administered may depend on the age of the patient, the particular condition to be treated, the frequency of administration, and the route of administration. The concentration of each of the active ingredients can range from about 0.001 percent to about 10 percent by weight of the composition. The preferred concentration of each of the estrogen analogue and androgen is from about 0.001 percent to about 1 percent by weight of the composition.

The “effective amount” or “pharmacologically effective amount” of active ingredients in a unit dose depends upon a number of factors. Included among those factors are the carrier when used, the tolerance for the active ingredients, the response elicited, and the number of unit dose administrations desired to be used. During treatment, the estrogen analogue and anabolic androgen may be administered to the eye by contacting the affected eye with a dosage in the range of about 0.0001 milligrams to about 10 milligrams per administration, the preferred dosage range being about 0.004 to about 4.0 milligrams per administration. The administrations may be continuous or repeated over a period of time.

The composition of the invention can take any of a number of forms depending on the carrier or vehicle used. For example, the composition may advantageously be a solution, suspension or ointment depending on the characteristics of the estrogen androgen and the vehicle. Suitable vehicles include aqueous, lipid, liposome, or polymer based solutions or suspensions.

The delivery vehicle for the combination therapy may be supplied as an over-the-counter artificial tear (solution) that can be used to provide temporary relief of dry eye symptoms. Such compositions may contain mucin-like substances (e.g., povidone and hydroxyethylcellulose) which mimic the action of the conjunctival mucus or render the surface of the eye more wetable. The vehicle helps keep the eye moist and assures that the tear film can spread easily and evenly over the eye surface.

The preferred vehicle for the combination 17-β-estradiol (as 3-phosphate disodium salt) and androgen (as the phosphate ester) has the following attributes:
a) a sterile, buffered isotonic solution.
b) contains mucin-like substances that tend to increase the contact time between the active drug substances (17-β-estradiol (as the 3-phosphate disodium salt) and androgenic agent and the eye surface.
c) free of benzalkonium chloride (if the phosphate esters are used), which is a cationic surfactant that is known to be incompatible in solutions with steroid sodium phosphate salts.

A preferred vehicle has the composition as shown in Table IV. A more preferred composition of the pharmaceutical carrier is: Dibasic sodium phosphate, USP 0.3%; Sodium Chloride, USP about 0.6%; Edetate disodium, USP 0.1%; Povidone K-15 or K-17, USP 0.37%; Poloxamer NF, 0.004%, PEG 0.12%; HEC NF, 0.2%, Purified water, USP, q.s to 100%; HCl or NaOH to adjust pH to pH 6-8. The preferred carrier may further comprise one or more preservatives selected from the group consisting of methylparaben (0.005-0.5 w/w %), proplyparaben (0.005-0.5 w/w %), benzalkonium chloride (0.005-1.0%) and phenoxyethanol (0.005-1.0 w/w %). Other modifications of the carrier solution may be made without departing from the scope of the pharmaceutically acceptable carrier of the present invention.

TABLE IV

| Compound | Concentration (w/w %) |
|---|---|
| Povidone (USP) | 0.05-2.0% |
| Hydroxyethylcellulose (USP) | 0.05-1.0% |
| Sodium chloride (USP) | 0.2-0.9% |
| Anhydrous sodium phosphate (Na₂HPO4) | 0.05-1.0% |
| (USP) | |
| Poloxamer NF | 0.001-05% |
| Polyethylene glycol | 0.05-1.0% |
| Disodium edate (USP) | 0.05-1.0% |
| dil. HCl or NaOH for pH adjustment | qs |
| purified water (USP) | qs |

In one embodiment, it is contemplated that a sterile, ophthalmic solution of 17-β-estradiol and the androgen can be comprised of a liposomal drug delivery system (Margalit R., Liposome-Mediated Drug Targetin in Topical and Regional Therapies, Crit. Rev. Ther. Drug Carrier Syst., 12(2-3):233-61 (1995)). Liposomal therapy has been successfully used in ophthalmology not only for pre- and postoperative antisepsis, but also for the treatment of bacterial and viral conjunctivitis and for prophylaxis against ophthalmia neonatorum (Reimer K, et al., Povidone-lodine Liposomes—An Overview, Dermatology, 195 Suppl. 2:93-9 (1997)). A Method for formulating such a product can be found in U.S. Pat. No. 5,662,931, which is incorporated herein by reference.

In an alternater embodiment, the composition comprises a sterile, ophthalmic suspension of 17-β-estradiol cypionate and androgen dissolved to form a 0.1% (by volume) solution in a vehicle which may in one embodiment take the form of a lipid based solution having a pH within the range of 4-8 with a preferred range of about 6-8.

In yet another embodiment, the composition comprises a sterile, ophthalmic solution of 17-β-estradiol (as 3-phosphate disodium salt) and androgen dissolved to form a 0.1% (by volume) solution in a vehicle which may in one embodiment take the form of an over-the-counter artificial tear solution. The concentration of the steroids in the vehicle is increased or decreased depending on the desired activity of the steroids.

In an alternate embodiment, the composition may take the form of a sterile ophthalmic ointment formulated to melt at body temperature. A suitable example of such a formulation may contain:

| Compound | Concentration (w/w %) |
|---|---|
| 17-β-estradiol (microcrystalline) | 0.001-1.0 |
| 17-α-methyl-17-β-hydroxy-2-oxa-5α- | 0.001-1.0 |
| androstan-3-one | |
| propyl paraben (USP) | 0.2 |
| Anhydrous liquid lanolin | 5.0 |
| mineral oil (USP) | 10.0 |
| white petrolatum (USP) | 84.6-84.7 |

In an alternate embodiment, the composition may take the form of a sterile aqueous ophthalmic suspension. A suitable example of such a formulation may contain:

| Compound | Concentration (w/w %) |
|---|---|
| 17-β-estradiol (microcrystalline) | 0.001-1.0 |
| 17-α-methyl-17-β-hydroxy-2-oxa-5α- | 0.001-1.0 |
| androstan-3-one | |
| Sodium chloride (USP) | 0.2-0.9% |
| Anhydrous sodium phosphate (Na₂HPO4) | 0.05-1.0% |
| (USP) | |
| Poloxamer NF | 0.001-0.05% |
| Polyethylene glycol | 0.05-1.0% |
| Disodium edetate (USP) | 0.05-1.0% |
| dil. HCl or NaOH for pH adjustment | qs |
| purified water (USP) | qs |
| Povidone, USP | 0.05-2.0% |
| Hydroxyethyl Cellulose NF | 0.05-1.0% |

In a more preferred embodiment, the composition comprises, on a weight basis:

| Compound | Concentration (w/w %) |
|---|---|
| 17-β-estradiol-3-phosphate | 0.001-1.0 |
| Androgen (as the phosphoester derivative) | 0.001-1.0 |
| Polyethylene glycol | 0.12 |
| Povidone (USP) | 0.37 |
| Hydroxyethylcellulose (USP) | 0.2 |
| Sodium chloride (USP) | 0.6 |
| Disodium edetate (USP) | 0.1 |
| Poloxamer NF | 0.004 |
| dil. HCl for pH adjustment | qs |
| purified water (USP) | qs |

The compositions may optionally contain one or more preservatives selected from the group consisting of methylparaben (0.005-0.5 w/w %), propylparaben (0.005-0.5 w/w %), benzalkonium chloride (0.005%-1.0%) and phenoxyethanol (0.005-1.0 w/w %).

### EXAMPLE 1

### Manufacturing and Packaging Procedure for a Preferred Composition of the Invention

A. Preparation of Estradiol

The method of synthesis of 17-β-estradiol-3 phosphate disodium is reported in Acta Chem. Scan. 12,1675-1689 (1958), which is incorporated herein by reference, and is briefly described as follows:

17-β-estradiol 17-acetate (Molecular Weight=314.4, Melting Point 220-224 ° C. and optical rotation 47°) is phosphorylated in the presence of concentrated ortho-phosphoric acid (H₃PO₄) with heat and refluxing to yield the intermediate 17-β-estradiol 3-phosphate 17-acetate. The latter compound is selectively hydrolyzed in the presence of sodium bicarbonate in aqueous alcohol to yield sodium acetate and 17-β-estradiol 3-phosphate disodium. The desired steroid phosphate ester is recrystallized from dilute alcohol. More information on the preparation and characteristics of 17-β-estradiol 3-phosphate is set forth in the article by Diczfalusy (22) which is incorporated herein by reference.

B. Preparation of Androgen

The synthesis and preparation of the androgens of the present invention are well known in the art and typically belong to the major structural subclasses of androgens, as disclosed in Vida (Vida, J. A., “Androgens and Anabolic Agents,” Academic Press, New York (1969)), hereby incorporated by reference. Preferred androgenic agents of this invention are those which have more anabolic, than virilizing effect, (e.g., oxandrolone possesses 322% of the anabolic and 24% of the androgenic activity of methyltestosterone (Wong et al. U.S. Pat. No. 5,766,242, (1998)).

C. Manufacturing Procedure

The preferred drug product used in our invention is manufactured and packaged as follows:
i) A calculated amount of androgen and 17-β-estradiol (as 3-phosphate disodium salt) on an “as is basis” is weighed on a suitable balance and transferred to a sufficient volume of vehicle.
ii) The composition is mixed until a solution of the steroids in the vehicle is obtained. (The pH of the solution may be adjusted to about pH 7 with dilute hydrochloric acid (HCl) or dilute sodium hydroxide (NaOH) if required). The composition is brought to final volume with additional vehicle and mixing.
iii) The compositiont is sterile filtered using an appropriate sterile filter assembly and a suitable syringe and filled directly into previously sterilized (see iv) 15 ml dropping bottles with a snap-tip dropper insert and polypropylene overcap (Wheaton Scientific, Millville, N.J. 08332). This portion of the operation is performed directly in front of a class 100 environment.
iv) Air blow Wheaton dropping bottles, inserts and caps are placed inside low density polyethylene sterilizing bag and the bag and contents are sterilized in a 3M ETO sterilizer unit for about 2 hours.

Compositions of the invention are preferably stored at controlled room temperature (15 to 30° C.) preferably at 22 to 24° C. as long as adequate physical stability (i.e., clarity of solution) is maintained. Otherwise storage under refrigeration (less than 10° C.) may be required.

D. Quality Assurance

The following quality control procedures are employed to assure identity, strength, quality and purity of the drug product:

Representative samples of finished compositiont are opened and examined for clarity of solution (clear, colorless to pale yellow solution, essentially free of foreign matter), pH content (not less than 7 and not more than 8) and a simple potency assay (absorbance read at 280 nanometers using 1 centimeter cells in a suitable spectrophotometer after diluting the drug product with alcohol or methanol to a suitable concentration). Comparison with the absorbance of a standard solution of 17-β-estradiol 3-phosphate disodium salt and the androgen is performed. Alternatively, HPLC assay may be used to compare the absorbance of the paraben-containing placebo versus the absorbance of the active drug formulation.

In yet an alternate embodiment, it is contemplated that the composition of said invention be free of any preservative compounds and said invention be provided to patient in a sterile single or similar package allowing no more than 7 days of use before the patient discards the package.

In another alternate embodiment, it is contemplated that the present invention utilizes an ocular insert means of delivering the combination steroids directly to the ocular surface and conjunctiva. Such delivery systems are well known in the art and are exemplified by the disclosure of U.S. Pat. No. 4,478,818, and hereby incorporated by reference.

In yet another alternate embodiment, it is contemplated that the present invention utilizes a thermosetting gel with a low sol-gel transition temperature as a method of delivering the combination steroid ingredients directly to the ocular surface and conjunctiva. Such delivery systems are well known in the art and are exemplified by the disclosure of U.S. Pat. No. 4,474,571, and also hereby incorporated by reference.

In yet another alternate embodiment, it is contemplated that the present invention utilizes the combination steroids as an encapsulated agent for introduction into the suprachoroid of the eye for therapeutic purposes. The administration of the steroids can be controlled and maintained for long periods of time, while ensuring the substantial absence of significant concentrations of steroids outside the site of administration. Examples of such materials and techniques are shown in the art (U.S. Pat. No. 4,853,224, U.S. Pat. No. 4,997,652, U.S. Pat. No. 5,164,188, U.S. Pat. No. 5,443,505, U.S. Pat. No. 5,766,242) and are hereby incorporated by reference.

### EXAMPLE 2

A liposome delivery vehicle is shown in the table below.

| Ingredient | Amount (w/w %) |
|---|---|
| 17-β-estradiol | Desired amount |
| 17-α-methyl-17-β-hydroxy-2-oxa-5α- | Desired amount |
| androstan-3-one | |
| Phosphotidylcholine | 3.0 |
| Phosphotidylserine | 3.0 |
| Carbomer (N.F.) | q.s. |
| Propylene glycol | 6.0 |
| C₁₂₋₁₅ benzoate | 2.0 |
| Emulsifying wax | 2.0 |
| Aminomethyl propanol | q.s. |
| Preservative (optional) | 1.0 |
| Purified water (U.S.P.) | q.s. |

Disperse the carbomer (a polymer of acrylic acid used in pharmaceutical preparations) in a portion of the purified water and heat to about 70° C. Add the 17-β-estradiol and 17-α-methyl-17-β-hydroxy-2-oxa-5α-androstan-3-one in the emulsifying wax, C₁₂₋₁₅ benzoate, both phospholipid derivatives and propylene glycol to about 70° C. Cool the solution to about 40° C. and adjust the pH of the solution to about pH 6.0 with the aminomethyl propanol. Add the preservative (if any) and add additional purified water to the final desired volume. While warm, filter under pressure through 0.2 μM membrane filter to form a sterile solution. Note that this method is described for example purposes and is not intended to show the only method that is possible.

### EXAMPLE 3

Prior to an application of a drug formulated in accordance with the present invention it is necessary to establish the presence of dry eye syndrome (KCS) in the test population and to follow its course under treatment. It is imperative that the diagnosis of dry eye syndrome be correct. Most often, KCS is diagnosed by use of the Schirmer test. The Schirmer test, however, is not always the most accurate test. It consists of taking a strip of filter paper 30 mm long and 5 mm in length and placing it in the patient's lower conjunctival sac. After 5 minutes, the length of paper that is moistened by the flow of tears is measured and used as an indicator of lacrimal fluid quantity. Factors such as temperature, humidity, lacrimal viscosity, types of filter paper used, batch variations between lots of paper, and other factors can affect the data produced by this test.

The diagnosis of dry eye syndrome in the present invention, can be made on the basis of one or more of the following tests. Microscopic evaluation of the tear film with particular attention to the marginal tear strip, viscosity and debris content of the precorneal tear film, and lid examination may be performed. Staining the ocular surface with Rose Bengal or Lissamine Green, vital dyes which indicate cellular damage, Schirmer testing, tear osmolarity, measurement of tear break-up time (TBUT), may also be used. In addition, the maturation index (a Papanicolaou stained sample of conjunctival epithelium) may also be performed.

In the case of post-menopausal women, menopause is confirmed with follicle stimulating hormone and luteinizing hormone serum determinants. Postmenopausal women with DES have been shown to have lower estradiol levels (mean E₂ estradiol levels of 3.47 picograms/milliliter), than that of normal postmenopausal women (mean E₂ estradiol levels of 16.05 picograms/milliliter) (U.S. Pat. No. Re. 34,578, col. 2, Ln. 56-59).

For example, one or two drops per eye given two to four times a day may be used, but application may also be more or less frequent. However, other alternative pharmaceutical modes of administration may be used—such as a slow release mode, or any other topical method, and the concentration may vary with individual response, as well as the treatment intervals and duration. Blood levels of the active hormone ingredients should also be determined and monitored.

It will be apparent to those skilled in the art that various modifications and variations can be made to the present invention without departing from the spirit or scope of the invention. Thus, it is intended that the present invention cover the modifications and variations of this invention provided they come within the scope of the appended claims and their equivalents. Accordingly, the invention is not limited by the embodiments described above which are presented as examples only, but can be modified in various ways within the scope of protection defined by the appended patent claims.

We claim:

1. A pharmaceutical composition for topical application in the treatment of dry eye syndrome comprising an estrogen analogue and an androgen in a pharmaceutically acceptable vehicle.

2. The pharmaceutical composition of claim 1, wherein said estrogen analogue is selected from 17-β-estradiol, beta-estradiol glucuromide, beta-estradiol hemisuccinate, beta-estradiol phosphate, beta-estradiol sulfate and their salts and esters.

3. The pharmaceutical composition of claim 2, wherein said estrogen analogue is 17-β-estradiol-3-phosphate disodium salt.

4. The pharmaceutical composition of claim 3, wherein said β-estradiol comprises from about 0.001 to about 1.0 weight percent of said composition.

5. The pharmaceutical composition of claim 1, wherein said androgen is selected from the group consisting of 17-α-methyl-17-β-hydroxy-2-oxa-5α-androstan-3-one, 4,5α-dihydrotestosterone derivatives, testosterone derivatives, 19-nortestosterone derivatives, and 17β-hydroxy-5α-androstane derivatives containing ring A unsaturation, their esters and their cationic or phosphorylated derivates.

6. The pharmaceutical composition of claim 1, wherein said androgen is selected from testosterone, dihydrotestosterone, fluoxymesterone, stanozolol, nortestosterone propionate, dehydroepiandrosterone, oxandrolone, oxymetholone, 5 alpha-androstan-17β-ol-3-oxime, 5 alpha-androstan-17 alpha-ol-3-one-acetate, (1) 2,(5 alpha)-androsten-17β-ol, 5 alpha-androstan-2 alpha-methyl-17β-ol-3-one, methyltestosterone and their derivatives and esters.

7. The pharmaceutical composition of claim 1, wherein said androgen is selected from 17-β-hydroxy-2-oxa-5α-androstan-3-one, 4,5α-dihydrotestosterone, and their nitrogenated or phosphorylated derivatives.

8. The pharmaceutical composition of claim 3, wherein said androgen is selected from 17-β-hydroxy-2-oxa-5α-androstan-3-one, 4,5α-dihydrotestosterone, and their nitrogenated or phosphorylated derivatives.

9. The pharmaceutical composition of claim 8, wherein said vehicle comprises, on a weight percent basis, about:

| | |
|---|---|
| Dibasic sodium phosphate | 0.05-1.0%; |
| Sodium Chloride | 0.2-0.9%; |
| Edetate disodium | 0.05-1.0%; |
| Povidone | 0.05-2.0%; |
| Poloxamer | 0.001-0.05%; |
| Polyethylene glycol | 0.05-1.0%; |
| Hydroxyethyl Cellulose | 0.05-1.0%; |
| Purified water | q.s to 100%; and |
| HCl or NaOH | to adjust pH to pH 6-8. |

10. The composition of claim 9, wherein said vehicle comprises, on a weight percent basis, about:

| | |
|---|---|
| Dibasic sodium phosphate | 0.3%; |
| Sodium Chloride | 0.6%; |
| Edetate disodium | 0.1%; |
| Povidone K-17 | 0.37%; |
| Poloxamer | 0.004%; |
| Polyethlyene glycol | 0.12%; |
| Hydroxyethyl Cellulose | 0.2%; |
| Purified water | q.s to 100%; |
| HCl or NaOH | to adjust pH to pH 6-8. |

11. The pharmaceutical composition of claim 1, wherein said androgen comprises from about 0.001 to about 1.0 weight percent of said composition.

12. The pharmaceutical composition of claim 4, wherein said androgen comprises from about 0.001 to about 1.0 weight percent of said composition.

13. A method for treating Dry Eye Syndrome (KCS) in a patient comprising, applying topically to the ocular surface or conjunctival tissue of the eye of said patient an effective amount of an estrogen analogue and an androgen.

14. The method of claim 13, wherein said estrogen analogue and said androgen are contained within a single composition.

15. The method of claim 14, wherein said estrogen analogue is selected from the group consisting of 17-β-estradiol, beta-estradiol glucuromide, beta-estradiol hemisuccinate, beta-estradiol phosphate, beta-estradiol sulfate and their salts and esters.

16. The method of claim 15, wherein said estrogen analogue is 17-β-estradiol-3-phosphate disodium salt.

17. The method of claim 16, wherein said β-estradiol comprises from about 0.001 to about 1.0 weight percent of said composition.

18. The method of claim 14, wherein said androgen is selected from the group consisting of 17-α-methyl-17-β-hydroxy-2-oxa-5α-androstan-3-one, 4,5α-dihydrotestosterone derivatives, testosterone derivatives, 19-nortestosterone derivatives, and 17β-hydroxy-5α-androstane derivatives containing ring A unsaturation, their esters and their cationic or phosphorylated derivates.

19. The method of claim 14, wherein said androgen is selected from testosterone, dihydrotestosterone, fluoxymesterone, stanozolol, nortestosterone propionate, dehydroepiandrosterone, oxandrolone;, oxymetholone, 5 alpha-androstan-17β-ol-3-oxime, 5 alpha-androstan-17 alpha-ol-3-one-acetate, (1) 2,(5 alpha)-androsten-17β-ol, 5 alpha-androstan-2 alpha-methyl-17β-ol-3-one, methyltestosterone and their derivatives and esters.

20. The method of claim 14, wherein said androgen is selected from 17-β-hydroxy-2-oxa-5α-androstan-3-one, 4,5α-dihydrotestosterone, and their nitrogenated or phosphorylated derivatives.

21. The method of claim 16, wherein said androgen is selected from 17-β-hydroxy-2-oxa-5α-androstan-3-one, 4,5α-dihydrotestosterone, and their nitrogenated or phosphorylated derivatives.

22. The method of claim 20, wherein said androgen comprises form about 0.001 to about 1.0 weight percent of said composition.

23. The method of claim 15, wherein said 17-β-estradiol or its derivatives and said androgen are dissolved or suspended in a lipid vehicle.

24. The method of claim 15, wherein said 17-β-estradiol or its derivatives and said androgen are water soluble esters and the vehicle in which they are applied consists essentially of an aqueous solution having a pH within the range of about 4 to about 8.

25. The method of claim 24, wherein said 17-β-estradiol and said androgens are dissolved or suspended in a delivery vehicle comprising, on a weight percenet basis:

| | |
|---|---|
| Dibasic sodium phosphate | 0.05-1.0%; |
| Sodium Chloride | 0.2-0.9%; |
| Edetate disodium | 0.05-1.0%; |
| Povidone, | 0.05-2.0%; |
| Poloxamer | 0.001-0.05%; |
| Polyethylene glycol | 0.05-1.0%; |
| Hydroxyethyl Cellulose | 0.05-1.0%; |
| Purified water | q.s to 100%; and |
| HCl or NaOH | to adjust pH to pH 6-8. |

26. The method of claim 22, wherein the delivery vehicle further comprises one or more preservatives selected from the group consisting of methylparaben, propylparaben, and phenoxyethanol.

27. A composition comprising 17-β-estradiol or its derivatives and an androgen having a concentration range of at least about 0.001% to less than 1.0% weight percent dissolved or suspended in a pharmaceutically acceptable liposomal vehicle.

28. A method of treating Dry Eye Syndrome comprising topically applying to the ocular surface or conjunctival tissue 17-β-estradiol or its derivatives and an androgeni dissolved or suspended in a polymeric composition biologically compatible with the eye.

29. The method of claim 28, wherein said polymeric composition is comprised of a thermosetting gel wherein the sol-gel transition temperature of said polymeric composition is room temperature or below and said polymeric composition is liquid at this temperature.

30. The method of claim 29, wherein said polymeric composition comprises a biodegradable controlled-release polymer.
